(19) United States
(12) Patent Application Publication
(10) Pub. No.: US 2005/0014170 A1
(43) Pub. Date: Jan. 20, 2005

(54) Effect of BDNF genotype on hippocampal function and verbal memory and risk for schizophrenia

(76) Inventors: Daniel R. Weinberger, Washington, DC (US); Michael F. Egan, Chevy Chase, MD (US); Bhaskar S. Kolachana, Oakton, VA (US); David Goldman, Potomac, MD (US); Joseph H. Callicott, Bethesda, MD (US); Terry E. Goldberg, Bethesda, MD (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/789,169

(22) Filed: Feb. 27, 2004

Related U.S. Application Data
(63) Continuation of application No. PCT/US02/28086, filed on Aug. 30, 2002.
(60) Provisional application No. 60/316,736, filed on Aug. 31, 2001.

(51) Int. Cl. C12Q 1/68

(52) U.S. Cl. 435/6

(57) ABSTRACT

The invention is related to the discovery that a met66val polymorphism in the gene for brain-derived neurotrophic factor (BDNF) is correlated with hippocampal function and verbal memory and risk for neuropsychiatric disorders such as schizophrenia.

### RELATED APPLICATIONS

This application is a continuation and claims the benefit of priority of International Application No. PCT/US02/28086 filed Aug. 30, 2002, designating the United States of America and published in English as WO 03/018847 on Mar. 6, 2003, which claims the benefit of priority of U.S. Provisional Application No. 60/316,736 filed Aug. 31, 2001, both of which are hereby expressly incorporated by reference in their entireties.

### FIELD OF THE INVENTION

The invention is related to the discovery that a met66val polymorphism in the gene for brain-derived neurotrophic factor (BDNF) is correlated with hippocampal function and verbal memory and risk for neuropsychiatric disorders such as schizophrenia.

### BACKGROUND OF THE INVENTION

The neurotrophins promote survival of neurons from both the central nervous system (CNS) and peripheral nervous system (PNS) in cell culture (for review, see, Reichardt L. F. & Farinas I. 1997 In: Molecular approaches to neural development, Cowan et al. eds., pp. 220-263, New York: Oxford UP). These four closely related proteins, nerve growth factor (NGF), brain-derived neurotrophic factor (BDNF), neurotrophin-3 (NT-3) and neurotrophin-4 (NT-4), interact with Trk receptor tyrosine kinases. TrkA is activated by NGF; TrkB is activated by BDNF and NT-4; and TrkC is activated by NT-3. Engagement of the Trk receptors results in activation of several intracellular signaling pathways, including ras, phosphatidylinositol-3 kinase, and phospholipase Cγ1, which promote survival and differentiation. All four neurotrophins also bind to the unrelated receptor p75NTR, which activates ceramide turnover and the jun kinase cascade, promoting either cell motility or apoptosis, depending on cell type. Significant attention has been directed toward the role of BDNF in synaptic transmission and plasticity in the hippocampus. An important new concept has emerged: neurotrophins may serve as a new class of neuromodulators that mediate activity-dependent modifications of neuronal connectivity and synaptic efficacy.

### SUMMARY OF THE INVENTION

The invention provides methods and kits for diagnosing and modulating hippocampal function and verbal memory and risk for neuropsychiatric disorders such as schizophrenia in an individual by determining the individual's BDNF genotype, and associating a met allele with impaired hippocampal function and verbal memory and risk for neuropsychiatric disorders such as schizophrenia (and a val allele with enhanced hippocampal function and verbal memory and protection from neuropsychiatric disorders such as schizophrenia).

### DETAILED DESCRIPTION OF THE PREFERRED EMBODIMENT

Neurotrophins are a family of structurally related proteins that include nerve growth factor (NGF), brain-derived neurotrophic factor (BDNF), neurotrophin-3 (NT-3), and NT-4. The signaling and biological functions of these molecules are mediated primarily by the Trk receptor tyrosine kinases. NGF binds to TrkA; BDNF and NT-4 to TrkB; and NT-3 to TrkC. According to the classic definition, neurotrophins, and indeed all neurotrophic factors, are endogenous signaling molecules that regulate the long-term survival and differentiation of specific populations of neurons during development, and the viability of neurons in adulthood. More recent studies have suggested an unexpected role for these factors in regulation of synaptic transmission and plasticity. Significant attention has been directed toward the role of BDNF in cortical structures, particularly the hippocampus. Numerous experiments have established that BDNF regulates hippocampal long-term potentiation (LTP), which implies a role in synaptic transmission and plasticity in the hippocampus.

Brain-derived neurotrophic factor (BDNF) has been cloned and shown to be homologous to NGF (Jones, K. R. & Reichardt, L. F. 1990 PNAS USA 87:8060-8064). BDNF is initially synthesized as a 247 amino acid protein precursor that is subsequently cleaved to yield the mature protein. The mature form of BDNF essentially corresponds to the C-terminal half of its precursor and constitutes 119 amino acids. The DNA sequence of human BDNF is given in GenBank accession no. M37762 and is shown in FIG. 2 of Jones and Reichardt, supra. The protein sequence of human BDNF can be deduced from GenBank accession no. M37762 (and is given in GenBank accession no. P23560) and is shown in FIG. 2 of Jones and Reichardt, supra, and compared to other members of the NGF family in FIG. 3 of Jones and Reichardt, supra. A single nucleotide polymorphism (SNP) from G to A in the DNA sequence results in the substitution of a methionine residue for a valine residue at amino acid position 66. Amino acids are numbered with position +1 assigned to the first residue in the precursor protein sequence for BDNF of 247 amino acids, e.g., using GenBank accession no. P23560. The average allele frequency of “G” has been estimated to be from 0.675 to over 0.8, and the average allele frequency of “A” has been estimated to be from 0.12 to 0.325.

As described herein, it has been discovered that a polymorphism in the gene for BDNF is correlated with hippocampal function and verbal memory and risk for neuropsychiatric disorders such as schizophrenia. In particular, it has been discovered that a single nucleotide polymorphism within the DNA sequence encoding the precursor protein sequence of 247 amino acids is correlated with impaired hippocampal function and verbal memory and risk for neuropsychiatric disorders such as schizophrenia. More particularly, a single nucleotide polymorphism from G to A resulting in the substitution of a methionine residue for a valine residue at amino acid position 66 (relative to the start of the precursor protein sequence) is correlated with impaired hippocampal function and verbal memory and risk for neuropsychiatric disorders such as schizophrenia. Conversely, a single nucleotide polymorphism from A to G resulting in the substitution of a valine residue for a methionine residue at amino acid position 66 (relative to the start of the precursor protein sequence) is correlated with enhanced hippocampal function and verbal memory and protection from neuropsychiatric disorders such as schizophrenia. This polymorphism resides within the amino acid precursor portion that is cleaved from the mature protein.

BDNF is a neurotrophin that mediates LTP and hippocampus-related spatial memory. The human BDNF gene contains at least one known nonconservative SNP, producing a met66val substitution. Schizophrenia, a complex genetic disorder, appears to involve hippocampal (HIP) abnormalities, including deficits in verbal memory, reduced HIP n-acetyl aspartate (NAA), a measure of neuronal integrity assessed with magnetic resonance spectroscopy (MRS), and abnormal patterns of hippocampal activation during memory tasks assessed with functional magnetic resonance imaging (fMRI). Verbal memory deficits, reduced HIP NAA, and abnormal hippocampal activation are also found in unaffected sibs of patients, suggesting a genetic trait related to susceptibility. We hypothesized that the met66val polymorphism would affect verbal memory and HIP NAA, thereby increasing risk for schizophrenia. We assessed verbal memory in 184 patients with schizophrenia, 283 siblings, and 101 controls. NAA was available for 110 subjects. The effect of genotype was significant across all groups for memory scores (p<0.008). The rarer met allele was associated with poorer performance. BDNF genotype had no effect on IQ or prefrontal cognitive measures. The met allele was also associated with reduced HIP NAA (p<0.07). In two separate cohorts studied with fMRI, subjects with a met allele had abnormal patterns of hippocampal activation while performing memory tasks, compared to val/val homozygote subjects. Furthermore, the frequency of the deleterious met allele was slightly higher in patients with schizophrenia (0.19) compared to controls (0.13) (p=0.05). In a transmission disequilibrium test (TDT) analysis, transmissions of met (n=38) vs. val (n=33) alleles did not differ significantly. These data indicate that BDNF met66val accounts for genetic variance in human hippocampal function and verbal memory. Furthermore, there is a modest increase in the deleterious met allele in patients with schizophrenia. This result indicates that the BDNF met allele increases risk for schizophrenia presumably by impairing HIP function.

Accordingly, the invention relates to a method for predicting the likelihood that an individual will have impaired (or enhanced) hippocampal function or verbal memory, or for aiding in the diagnosis of risk of (or protection from) a neuropsychiatric disorder, i.e., schizophrenia, schizoaffective disorder, and other psychotic and mental disorders involving impaired memory, comprising the steps of obtaining a DNA sample from an individual to be assessed and determining the presence or absence of a single nucleotide polymorphism from G to A resulting in the substitution of a methionine residue for a valine residue at amino acid position 66 (relative to the start of the precursor protein sequence for BDNF). A single nucleotide polymorphism from G to A resulting in the substitution of a methionine residue for a valine residue at amino acid position 66 (relative to the start of the precursor protein sequence) is correlated with impaired hippocampal function and verbal memory and risk for neuropsychiatric disorders such as schizophrenia. Conversely, a single nucleotide polymorphism from A to G resulting in the substitution of a valine residue for a methionine residue at amino acid position 66 (relative to the start of the precursor protein sequence) is correlated with enhanced hippocampal function and verbal memory and protection from neuropsychiatric disorders such as schizophrenia. In a particular embodiment, the individual is an individual at risk for development of impaired hippocampal function or verbal memory or neuropsychiatric disorders such as schizophrenia. In another embodiment the individual exhibits clinical symptomatology associated with impaired hippocampal function or verbal memory or neuropsychiatric disorders such as schizophrenia. In still another embodiment, the individual has been clinically diagnosed as having impaired hippocampal function or verbal memory or neuropsychiatric disorders such as schizophrenia. Neuropsychiatric disorders include schizophrenia, schizoaffective disorder, and other psychotic and mental disorders involving impaired memory. Psychotic and mental disorders involving impaired memory include Alzheimer's Disease, head injuries, and normal ageing.

The genetic material to be assessed can be obtained from any nucleated cell from the individual. For assay of genomic DNA, virtually any biological sample (other than pure red blood cells) is suitable. For example, convenient tissue samples include whole blood, semen, saliva, tears, urine, fecal material, sweat, skin and hair. For assay of cDNA or mRNA, the tissue sample must be obtained from an organ in which the target nucleic acid is expressed. For example, cells from the central nervous system (such as cells of the hippocampus), neural crest-derived cells, skin, heart, lung and skeletal muscle are suitable sources for obtaining cDNA for the BDNF gene. Neural crest-derived cells include, for example, melanocytes and keratinocytes.

Many of the methods described herein require amplification of DNA from target samples. This can be accomplished by e.g., PCR. See generally: PCR Technology: Principles and Applications for DNA Amplification ed. H. A. Erlich, Freeman Press, NY, N.Y., 1992; PCR Protocols: A Guide to Methods and Applications eds. Innis, et al., Academic Press, San Diego, Calif., 1990; Mattila et al. 1991 Nucleic Acids Res 19:4967; Eckert et al. 1991 PCR Methods and Applications 1:17; PCR eds. McPherson et al., IRL Press, Oxford; and U.S. Pat. No. 4,683,202.

Other suitable amplification methods include the ligase chain reaction (LCR) (see, Wu & Wallace 1989 Genomics 4:560; Landegren et al. 1988 Science 241:1077) transcription amplification (Kwoh et al. 1989 PNAS USA 86:1173), and self-sustained sequence replication (Guatelli et al. 1990 PNAS USA 87:1874) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The single nucleotide polymorphism from G to A resulting in the substitution of a methionine residue for a valine residue at amino acid position 66 (relative to the start of the precursor protein sequence for BDNF) can be identified by a variety of methods, such as Southern analysis of genomic DNA; direct mutation analysis by restriction enzyme digestion; Northern analysis of RNA; denaturing high pressure liquid chromatography (DHPLC); gene isolation and sequencing; hybridization of an allele-specific oligonucleotide with amplified gene products; single base extension (SBE); or analysis of the BDNF protein. A sampling of suitable procedures are discussed below in turn.

### Allele-Specific Probes

The design and use of allele-specific probes for analyzing polymorphisms is described by e.g., Saiki et al. 1986 Nature 324:163-166; Dattagupta, EP 235,726; Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Hybridizations are usually performed under stringent conditions, for example, at a salt concentration of no more than 1 M and a temperature of at least 25° C. For example, conditions of 5× SSPE (750 mM NaCl, 50 mM NaPhosphate, 5 mM EDTA, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

### Tiling Arrays

The polymorphisms can also be identified by hybridization to nucleic acid arrays, some examples of which are described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

### Allele-Specific Primers

An allele-specific primer hybridizes to a site on target DNA overlapping a polymorphism and only primes amplification of an allelic form to which the primer exhibits perfect complementarity. See, Gibbs 1989 Nucleic Acid Res 17:2427-2448. This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

### Direct-Sequencing

The direct analysis of the sequence of polymorphisms of the present invention can be accomplished using either the dideoxy chain termination method or the Maxam Gilbert method (see, Sambrook et al. 1989 Molecular Cloning, A Laboratory Manual, 2^{nd} ed., CSHP, New York; Zyskind et al. 1988 Recombinant DNA Laboratory Manual, Acad. Press.)

### Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution (Erlich, ed. 1992 PCR Technology, Principles and Applications for DNA Amplification, W. H. Freeman and Co, New York, Chapter 7).

### Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al. 1989 PNAS USA 86:2766-2770. Amplified PCR products can be generated as described above, and heated or otherwise denatured, to form single-stranded amplification products. Single-stranded nucleic acids may refold or form secondary structures which are partially dependent on the base sequence. The different electrophoretic mobilities of single-stranded amplification products can be related to base-sequence differences between alleles of target sequences.

### Other Assays

The polymorphism of the invention may contribute to the susceptibility of an individual to impaired hippocampal function and verbal memory and risk for neuropsychiatric disorders such as schizophrenia in different ways. The polymorphism may contribute to phenotype by affecting gene transcription or processes related to translation, such as stability of the mRNA template. The polymorphism may also contribute to phenotype by affecting protein structure. By altering amino acid sequence, the polymorphism may alter the function of the encoded protein. The polymorphism may exert phenotypic effects indirectly via influence on replication, transcription, and translation. For example, the substitution of a methionine for a valine in the precursor portion of the BDNF protein may create an alternative translation start site which alters the length of the gene product and the precursor portion itself. Alteration of the length of the precursor protein may affect cleavage of the mature protein either positively or negatively. Alternatively, the presence of the variant amino acid may alter the properties of the precursor protein so as to alter activity dependent expression or cleavage of the precursor protein. More than one phenotypic trait may be affected. For example, other neuropsychiatric disorders which are believed to be alternate expressions of a schizophrenia genotype may also be affected by the BDNF polymorphism described herein. Additionally, the described polymorphism may predispose an individual to a distinct mutation that is causally related to a certain phenotype, such as increased or reduced hippocampal n-acetyl aspartate (NAA), a measure of neuronal integrity assessed with MRS. The discovery of the polymorphism and its correlation with hippocampal function and verbal memory and risk for neuropsychiatric disorders such as schizophrenia facilitates biochemical analysis of the variant and the development of assays to characterize the variant and to screen for pharmaceuticals that interact directly with one or another form of the protein.

In another embodiment, the invention relates to pharmaceutical compositions comprising a variant or reference BDNF gene product. As used herein, a reference BDNF gene product is intended to mean gene products which are encoded by the val allele of the BDNF gene and includes, but is not limited to, the complete (uncleaved), cleaved, and precursor portion of the reference BDNF gene product. A variant BDNF gene product is intended to mean gene products which are encoded by the met allele of the BDNF gene and includes, but is not limited to, the complete (uncleaved), cleaved, and precursor portion of the variant BDNF gene product. In one embodiment, the gene product is a protein comprising amino acids 1 through 247 of a reference BDNF gene product, or a functional portion thereof, for use in the treatment of impaired hippocampal function and verbal memory and risk for neuropsychiatric disorders such as schizophrenia. The invention further relates to the use of compositions (i.e., agonists) which in some manner alter, enhance, or increase the activity of a protein comprising amino acids 1 through 247 of the reference BDNF gene product, or a functional portion thereof, or the duration of action of the functional portion of the BDNF gene product, or its binding to its receptor or the subsequent effects of its receptor for use in the treatment of impaired hippocampal function and verbal memory and risk for neuropsychiatric disorders such as schizophrenia. The invention also relates to the use of compositions (i.e., antagonists) which in some manner alter, reduce, or decrease the activity of a protein comprising amino acids 1 through 247 of the variant BDNF gene product, or a functional portion thereof, or the duration of action of the functional portion of the BDNF gene product, or its binding to its receptor or the subsequent effects of its receptor for use in the treatment of impaired hippocampal function and verbal memory and risk for neuropsychiatric disorders such as schizophrenia.

In addition to substantially full-length polypeptides expressed by reference or variant genes, the present invention includes biologically active fragments of the polypeptides, or analogs thereof, including organic molecules which simulate the interactions of the polypeptides. Biologically active fragments include any portion of the full-length polypeptide which confers a biological function on the reference or variant gene product, including ligand binding, tyrosine phosphorylation and antibody binding. Ligand binding includes binding by nucleic acids, proteins or polypeptides, small biologically active molecules, or large cellular structures.

Polyclonal and/or monoclonal antibodies that specifically bind one form of the gene product but not to the other form of the gene product are also provided. Antibodies are also provided that bind a portion of either the variant or the reference gene product that contains the polymorphic site. Antibodies can be made by injecting mice or other animals with, for example, the variant or the reference gene product or peptide fragments thereof comprising the met66val portion. The peptide fragments can be synthetically produced or produced in a suitable host cell expressing a nucleic acid encoding said peptide. Monoclonal antibodies are screened as are described, for example, in Harlow & Lane 1988 Antibodies, A Laboratory Manual, Cold Spring Harbor Press, New York; Goding 1986 Monoclonal antibodies, Principles and Practice 2nd ed. Academic Press, New York. Monoclonal antibodies are tested for specific immunoreactivity with, for example, a variant gene product and lack of immunoreactivity to the corresponding reference gene product. In another embodiment, antibodies are produced and tested for specific immunoreactivity to the reference gene product and lack of immunoreactivity to the variant gene product. These antibodies are useful in diagnostic assays for detection of the variant or reference form, or as an active ingredient in a pharmaceutical composition.

The invention further relates to a method of predicting the likelihood than an individual will have impaired (or enhanced) hippocampal function or verbal memory, or for aiding in the diagnosis of risk of (or protection from) a neuropsychiatric disorder, i.e., schizophrenia, schizoaffective disorder, and other psychotic and mental disorders involving impaired memory. The method comprises obtaining a biological sample containing the precursor BDNF protein or relevant portion thereof from the individual and determining the amino acid present at amino acid position +66 relative to the first amino acid of the precursor protein. As used herein, the term “fragment thereof” of the precursor BDNF protein is intended to encompass any portion of the protein which comprises the polymorphic amino acid position. The presence of the variant amino acid, methionine, at this position is indicative of impaired hippocampal function and verbal memory and risk for neuropsychiatric disorders such as schizophrenia. Conversely, the presence of the reference amino acid, valine, at this position is indicative of enhanced hippocampal function and verbal memory and protection from neuropsychiatric disorders such as schizophrenia. In a particular embodiment, the individual is an individual at risk for development of impaired hippocampal function or verbal memory or neuropsychiatric disorders such as schizophrenia. In another embodiment the individual exhibits clinical symptomatology associated with impaired hippocampal function or verbal memory or neuropsychiatric disorders such as schizophrenia. In one embodiment, the individual has been clinically diagnosed as having impaired hippocampal function or verbal memory or risk for neuropsychiatric disorders such as schizophrenia.

In this embodiment of the invention, the biological sample contains protein molecules from the test subject. As described above for BDNF cDNA or mRNA, suitable sources for the biological sample are any tissue or bodily fluid that is expected to express or contain precursor BDNF protein or the precursor portion of BDNF can be used. In vitro techniques for detection of protein of interest include enzyme linked immunosorbent assays (ELISAs), Western blots, immunoprecipitations and immunofluorescence. Furthermore, in vivo techniques for detection of protein include introducing into a subject a labeled anti-protein antibody. For example, the antibody can be labeled with a radioactive marker whose presence and location in a subject can be detected by standard imaging techniques. Polyclonal and/or monoclonal antibodies that specifically bind to variant gene products but not to corresponding reference gene products, and vice versa, are also provided. Antibodies can be made as described above. These antibodies are useful in diagnostic assays for detection of the variant or reference form, or as an active ingredient in a pharmaceutical composition.

The invention also encompasses kits for detecting the presence of proteins or nucleic acid molecules of the invention in a biological sample. For example, the kit can comprise a labeled compound or agent capable of detecting protein or mRNA (or cDNA produced from the mRNA) in a biological sample; means for determining the identity of the met66val genotype in the protein or mRNA in the sample; and means for comparing the identity of the protein or mRNA in the sample with a suitable standard. The kit can also comprise control samples for use as standards, representing individuals homozygous for the reference or variant nucleotide in the case of analyzing nucleic acid, or the reference or variant amino acid in the case of analyzing proteins, or representing a heterozygous individual. For the detection of the reference or variant precursor portion of BDNF, the kit can contain antibodies specific for either the reference or the variant of BDNF together with suitable reagents to detect antibody binding to its target antigen. The compound or agent can be packaged in a suitable container. The kit can further comprise instructions for using the kit to detect protein or nucleic acid.

The invention further pertains to compositions, e.g., vectors, comprising a nucleotide sequence encoding variant or reference BDNF gene product. In one embodiment, the gene product is a polypeptide comprising amino acids +1 through +247 of the variant or reference BDNF gene product, or a functional portion thereof, for use in the modulation of hippocampal function or verbal memory or neuropsychiatric disorders such as schizophrenia. In another embodiment, the gene product is a polypeptide comprising amino acids +1 through +247 of the reference BDNF gene product, or a functional portion thereof, for use in the treatment of impaired hippocampal function or verbal memory or neuropsychiatric disorders such as schizophrenia. For example, reference genes can be expressed in an expression vector in which a reference gene is operably linked to a native or other promoter. Usually, the promoter is a eukaryotic promoter for expression in a mammalian cell. The transcription regulation sequences typically include a heterologous promoter and optionally an enhancer which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular construction and the target host. Suitable means include fusion, conjugation, transfection, transduction, electroporation or injection, as described in Sambrook et al. 1989 Molecular Cloning, A Laboratory Manual 2^{nd} ed. CSHP, New York. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the gene product to produce an appropriate mature polypeptide. Processing includes glycosylation, ubiquitination, disulfide bond formation, general post-translational modification, and the like.

It is also contemplated that cells can be engineered to express the variant or reference BDNF allele of the invention by gene therapy methods. For example, DNA encoding the reference BDNF gene product, or an active fragment or derivative thereof, can be introduced into an expression vector, such as a viral vector, and the vector can be introduced into appropriate cells in an animal. In such a method, the cell population can be engineered to inducibly or constitutively express active reference BDNF gene product. In a preferred embodiment, the vector is delivered to the bone marrow, for example as described in Corey et al. 1989 Science 244:1275-1281.

The invention further provides transgenic nonhuman animals capable of expression an exogenous (i.e., human) variant or reference BDNF gene and/or having one or both alleles of an endogenous BDNF gene inactivated. Expression of an exogenous variant or reference gene is usually achieved by operably linking the gene to a promoter and optionally an enhancer, and microinjecting the construct into a zygote. See Hogen et al. in: Manipulating the Mouse Embryo, A Laboratory Manual Cold Spring Harbor Laboratory. Inactivation of endogenous BDNF genes can be achieved by forming a transgene in which a cloned BDNF gene is inactivated by insertion of a positive selection marker. See, Capecchi 1989 Science 244:1288-1292. The transgene is then introduced into an embryonic stem cell, where it undergoes homologous recombination with an endogenous variant gene. Mice and other rodents are preferred animals. Such animals provide useful drug screening systems.

### Screening Assays for Compounds that Modulate BDNF and BDNF Receptor Expression or Activity

The activity of BDNF and its receptor TrkB (GDB ID: 127898) can be assessed using a variety of in vitro and in vivo assays to determine functional, chemical, and physical effects, e.g., measuring ligand binding (e.g., radioactive ligand binding), second messengers (e.g., cAMP, cGMP, IP₃, DAG, or Ca²⁺), ion flux, tyrosine phosphorylation levels, transcription levels, neurotransmitter levels, and the like. Furthermore, such assays can be used to test for inhibitors and activators of BDNF interaction with BDNF receptor family members. Modulators can also be genetically altered versions of BDNF receptors. Such modulators of BDNF and BDNF receptors are useful for modulating hippocampal function or verbal memory or neuropsychiatric disorders such as schizophrenia.

BDNF and BDNF receptor (trkB) of an assay described herein have amino acid sequence identify to the sequences given in the citations supra. Alternatively, the BDNF and BDNF receptor of the assay will have amino acid sequence identity at least 60%, optionally at least 70% to 85%, optionally at least 90-95%. Optionally, the polypeptide of the assays will comprise a domain of a BDNF receptor protein, such as an extracellular domain, transmembrane domain, cytoplasmic domain, ligand-binding domain, subunit-association domain, active site, and the like. Either the BDNF receptor or a domain thereof can be covalently linked to a heterologous protein to create a chimeric protein used in assays described herein.

Modulators of BDNF receptor activity are tested using BDNF receptor polypeptides as described above, either recombinant or naturally occurring. The protein can be isolated, expressed in a cell, expressed in a membrane derived from a cell, expressed in tissue or in an animal, either recombinant or naturally occurring. For example, hippocampal slices, dissociated cells, transformed cells, or membranes can be used. Modulation is tested using one of the in vitro or in vivo assays described herein. BDNF receptor-mediated signal transduction can also be examined in vitro with soluble or solid state reactions, using a full-length BDNF receptor or a chimeric molecule such as an extracellular domain or transmembrane domain, or combination thereof, or a BDNF receptor covalently linked to a heterologous signal transduction domain, or a heterologous extracellular domain and/or transmembrane domain covalently linked to the transmembrane and/or cytoplasmic domain of a BDNF receptor. Furthermore, ligand-binding domains of the candidate of interest can be used in vitro in soluble or solid state reactions to assay for ligand binding. In numerous embodiments, a chimeric receptor will be made that comprises all or part of a BDNF receptor polypeptide.

Ligand binding to a BDNF receptor protein, a domain, or chimeric protein can be tested in solution, in a bilayer membrane, attached to a solid phase, in a lipid monolayer, or in vesicles. Binding of a modulator can be tested using, e.g., changes in spectroscopic characteristics (e.g., fluorescence, absorbance, refractive index) hydrodynamic (e.g., shape), chromatographic, or solubility properties.

### Assays for Modulators of BDNF and BDNF Receptors

The following assays are designed to identify compounds that interact with (e.g., bind to) BDNF receptor (including, but not limited to an extracellular domain “ECD” or a cytoplasmic domain “CD” or a transmembrane domain “TMD” of BDNF receptor), compounds that interact with (e.g., bind to) intracellular proteins that interact with BDNF receptor (including, but not limited to, a TMD or a CD of BDNF receptor), compounds that interfere with the interaction of BDNF receptor with transmembrane or intracellular proteins involved in BDNF receptor-mediated signal transduction, and to compounds which modulate the activity of BDNF receptor gene (i.e., modulate the level of BDNF receptor gene expression) or modulate the level of BDNF receptor activity. Assays may additionally be utilized which identify compounds which bind to BDNF receptor gene regulatory sequences (e.g., promoter sequences) and which may modulate BDNF receptor gene expression. See e.g., Platt, K. A. 1994 J Biol Chem 269:28558-28562.

The compounds which may be screened in accordance with the invention include, but are not limited to peptides, antibodies and fragments thereof, and other organic compounds (e.g., peptidomimetics, small molecules) that bind to one or more ECDs of the BDNF receptor and either mimic the activity triggered by the natural ligand (i.e., agonists) or inhibit the activity triggered by the natural ligand (i.e., antagonists); as well as peptides, antibodies or fragments thereof, and other organic compounds that mimic the ECD of the BDNF receptor (or a portion thereof) and bind to and “neutralize” natural ligand, BDNF.

Such compounds may include, but are not limited to, peptides such as, for example, soluble peptides, including but not limited to members of random peptide libraries; (see, e.g., Lam, K. S. et al. 1991 Nature 354:82-84; Houghten, R. et al. 1991 Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang, Z. et al. 1993 Cell 72:767-778), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), and small organic or inorganic molecules.

Other compounds which can be screened in accordance with the invention include but are not limited to small organic molecules that are able to gain entry into an appropriate cell and affect the expression of the BDNF receptor gene or some other gene involved in the BDNF receptor signal transduction pathway (e.g., by interacting with the regulatory region or transcription factors involved in gene expression); or such compounds that affect the activity of the BDNF receptor or the activity of some other intracellular factor involved in the BDNF receptor signal transduction pathway.

Computer modeling and searching technologies permit identification of compounds, or the improvement of already identified compounds, that can modulate BDNF receptor expression or activity. Having identified such a compound or composition, the active sites or regions are identified. Such active sites might typically be ligand binding sites, such as the interaction domains of BDNF with BDNF receptor itself. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures, such as high resolution electron microscopy. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential BDNF receptor modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of BDNF, BDNF receptor, and related transduction and transcription factors will be apparent to those of skill in the art.

Examples of molecular modeling systems are the CHARMM and QUANTA programs (Polygen Corporation, Waltham, Mass.). CHARMM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen, et al. 1988 Acta Pharmaceutical Fennica 97:159-166; Ripka, 1988 New Scientist 54-57; McKinaly & Rossmann 1989 Annu Rev Pharmacol Toxicol 29:111-122; Perry & Davies 1989 OSAR: Quantitative Structure-Activity Relationships in Drug Design pp. 189-193 Alan R. Liss, Inc.; Lewis & Dean 1989 Proc R Soc Lond 236:125-140 and 141-162; and, with respect to a model receptor for nucleic acid components, Askew, et al. 1989 J Am Chem Soc 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds which are inhibitors or activators.

Compounds identified via assays such as those described herein are useful for designing modulators of hippocampal function or verbal memory or neuropsychiatric disorders such as schizophrenia.

### In Vitro Screening Assays for Compounds that Bind to BDNF and BDNF Receptor

In vitro systems may be designed to identify compounds capable of interacting with (e.g., binding to) BDNF and BDNF receptor (including, but not limited to, an ECD, or a TMD, or a CD of BDNF receptor). Compounds identified may be useful, for example, in modulating the activity of BDNF and BDNF receptors, may be utilized in screens for identifying compounds that disrupt or augment normal BDNF and BDNF receptor interactions, or may in themselves disrupt or augment such interactions.

The principle of the assays used to identify compounds that bind to BDNF and BDNF receptor involves preparing a reaction mixture of the BDNF receptor (or BDNF) and a test compound under conditions and for a time sufficient to allow the two components to interact and bind, thus forming a complex which can be removed and/or detected in the reaction mixture. The BDNF receptor (or BDNF) species used can vary depending upon the goal of the screening assay. For example, where agonists or antagonists of the BDNF are sought, the full length BDNF receptor, or a soluble truncated BDNF receptor, e.g., in which a TMD and/or a CD is deleted from the molecule, a peptide corresponding to an ECD or a fusion protein containing a BDNF receptor ECD fused to a protein or polypeptide that affords advantages in the assay system (e.g., labeling, isolation of the resulting complex, etc.) can be utilized. Where compounds that interact with the cytoplasmic domain are sought to be identified, peptides corresponding to a BDNF receptor CD and fusion proteins containing a BDNF receptor CD can be used.

The screening assays can be conducted in a variety of ways. For example, one method to conduct such an assay would involve anchoring the BDNF receptor (or BDNF) protein, polypeptide, peptide or fusion protein or the test substance onto a solid phase and detecting BDNF receptor (or BDNF)/test compound complexes anchored on the solid phase at the end of the reaction. In one embodiment of such a method, the BDNF receptor (or BDNF) reactant may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for BDNF receptor (or BDNF) protein, polypeptide, peptide or fusion protein or the test compound to anchor any complexes formed in solution, and a labeled antibody specific for the other component of the possible complex to detect anchored complexes.

Alternatively, cell-based assays, membrane vesicle-based assays and membrane fraction-based assays can be used to identify compounds that interact with BDNF receptor. To this end, cell lines that express BDNF receptor, or cell lines (e.g., COS cells, CHO cells, fibroblasts, etc.) have been genetically engineered to express BDNF receptor (e.g., by transfection or transduction of BDNF receptor DNA) can be used. Interaction of the test compound with, for example, an ECD or a CD of BDNF receptor expressed by the host cell can be determined by comparison or competition with BDNF.

A BDNF receptor may be employed in a screening process for compounds which bind the receptor and which activate (agonists) or inhibit activation (antagonists) of the receptor. Thus, BDNF receptors may also be used to assess the binding of small molecule substrates and ligands in, for example, cells, cell-free preparations, chemical libraries, and natural product mixtures. These substrates and ligands may be natural substrates and ligands or may be structural or functional mimetics. See, Coligan et al. 1991 Current Protocols in Immunology 1 (2): Chapter 5.

In general, such screening procedures involve providing appropriate cells which express a BDNF receptor on the surface thereof. Such cells include cells from mammals, insects, yeast, and bacteria. In particular, a polynucleotide encoding the BDNF receptor is employed to transfect cells to thereby express a BDNF receptor. The expressed receptor is then contacted with a test compound to observe binding, stimulation or inhibition of a functional response.

One such screening procedure involves the use of melanophores that are transfected to express a BDNF receptor. Such a screening technique is described in PCT WO 92/01810, published Feb. 6, 1992. Such an assay may be employed to screen for a compound which inhibits activation of a BDNF receptor by contacting the melanophore cells which encode the receptor with both a receptor ligand, such as BDNF, and a compound to be screened. Inhibition of the signal generated by the ligand indicates that a compound is a potential antagonist for the receptor, i.e., inhibits activation of the receptor.

The technique may also be employed for screening of compounds which activate a BDNF receptor by contacting such cells with compounds to be screened and determining whether such compound generates a signal, i.e., activates the receptor.

Other screening techniques include the use of cells which express a BDNF receptor (for example, transfected CHO cells) in a system which measures extracellular pH changes caused by receptor activation. In this technique, compounds may be contacted with cells expressing a BDNF receptor. A second messenger response, e.g., signal transduction or pH changes, is then measured to determine whether the potential compound activates or inhibits the receptor.

Another screening technique involves expressing a BDNF receptor in which the receptor is linked to phospholipase C or D. Representative examples of such cells include, but are not limited to, endothelial cells, smooth muscle cells, and embryonic kidney cells. The screening may be accomplished as hereinabove described by detecting activation of the receptor or inhibition of activation of the receptor from the phospholipase second signal.

Another method involves screening for compounds which are antagonists, and thus inhibit activation of a BDNF receptor by determining inhibition of binding of labeled ligand, such as BDNF, to cells which have the receptor on the surface thereof, or cell membranes containing the receptor. Such a method involves transfecting a eukaryotic cell with a DNA encoding a BDNF receptor such that the cell expresses the receptor on its surface (or using a eukaryotic cell that expresses the receptor on its surface). The cell is then contacted with a potential antagonist in the presence of a labeled form of a ligand, such as BDNF. The ligand can be labeled, e.g., by radioactivity. The amount of labeled ligand bound to the receptors is measured, e.g., by measuring radioactivity associated with transfected cells or membrane from these cells. If the compound binds to the receptor, the binding of labeled ligand to the receptor is inhibited as determined by a reduction of labeled ligand that binds to the receptors.

Another such screening procedure involves the use of eukaryotic cells, which are transfected to express the BDNF receptor, or use of eukaryotic cells that express the BDNF receptor on their surface. The cells are loaded with an indicator dye that produces a fluorescent signal when bound to calcium, and the cells are contacted with a test substance and a receptor agonist, such as BDNF. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist (or agonist) for the receptor.

Another such screening procedure involves use of eukaryotic cells, which are transfected to express the BDNF receptor (or use of eukaryotic cells that express the BDNF receptor), and which are also transfected with a reporter gene construct that is coupled to activation of the receptor (for example, luciferase or beta-galactosidase behind an appropriate promoter). The cells are contacted with a test substance and a receptor agonist, such as BDNF, and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the receptor.

Another such screening technique for antagonists or agonists involves introducing RNA encoding a BDNF receptor into Xenopus oocytes to transiently express the receptor. The receptor expressing oocytes are then contacted with a receptor ligand, such as BDNF, and a compound to be screened. Inhibition or activation of the receptor is then determined by detection of a signal, such as, cAMP, calcium, proton, or other ions.

Another such technique of screening for antagonists or agonists involves determining inhibition or stimulation of BDNF receptor-mediated cAMP and/or adenylate cyclase accumulation or diminution. Such a method involves transiently or stably transfecting a eukaryotic cell with a BDNF receptor to express the receptor on the cell surface (or using a eukaryotic cell that expresses the BDNF receptor on its surface). The cell is then exposed to potential antagonists in the presence of ligand, such as BDNF. The amount of cAMP accumulation is then measured, for example, by radio-immuno or protein binding assays (for example using Flashplates or a scintillation proximity assay). Changes in cAMP levels can also be determined by directly measuring the activity of the enzyme, adenylyl cyclase, in broken cell preparations. If the potential antagonist binds the receptor, and thus inhibits BDNF receptor binding, the levels of BDNF receptor-mediated cAMP, or adenylate cyclase activity, will be reduced or increased.

### Assays for Intracellular Proteins that Interact with BDNF and BDNF Receptor

Any method suitable for detecting protein-protein interactions may be employed for identifying transmembrane proteins or intracellular proteins that interact with BDNF and BDNF receptor. Among the traditional methods which may be employed are co-immunoprecipitation, crosslinking and co-purification through gradients or chromatographic columns of cell lysates or proteins obtained from cell lysates and BDNF and BDNF receptor to identify proteins in the lysate that interact with BDNF and BDNF receptor. For these assays, the BDNF receptor component used can be a full length BDNF and BDNF receptor, a soluble derivative lacking the membrane-anchoring region (e.g., a truncated BDNF receptor in which all TMDs are deleted resulting in a truncated molecule containing ECDs fused to CDs), a peptide corresponding to a CD or a fusion protein containing a CD of BDNF receptor. Once isolated, such an intracellular protein can be identified and can, in turn, be used, in conjunction with standard techniques, to identify proteins with which it interacts. For example, at least a portion of the amino acid sequence of an intracellular protein which interacts with BDNF and BDNF receptor can be ascertained using techniques well known to those of skill in the art, such as via the Edman degradation technique. (See, e.g., Creighton 1983 Proteins: Structures and Molecular Principles W. H. Freeman & Co., N.Y., pp. 34-49). The amino acid sequence obtained may be used as a guide for the generation of oligonucleotide mixtures that can be used to screen for gene sequences encoding such intracellular proteins. Screening may be accomplished, for example, by standard hybridization or PCR techniques. Techniques for the generation of oligonucleotide mixtures and the screening are well known. (See, e.g., Ausubel et al., 1989 Current Protocols in Molecular Biology Green Publishing Associates and Wiley Interscience, N.Y.; and Innis, M. et al. eds. 1990 PCR Protocols: A Guide to Methods and Applications Academic Press, Inc., New York).

Additionally, methods may be employed which result in the simultaneous identification of genes, which encode the transmembrane or intracellular proteins interacting with BDNF and BDNF receptor. These methods include, for example, probing expression libraries, in a manner similar to the well known technique of antibody probing of λgt11 libraries, using labeled BDNF receptor (or BDNF) protein, or a BDNF receptor (or BDNF) polypeptide, peptide or fusion protein, e.g., a BDNF receptor polypeptide or BDNF receptor domain fused to a marker (e.g., an enzyme, fluor, luminescent protein, or dye), or an Ig-Fc domain.

One method that detects protein interactions in vivo, the two-hybrid system, is described in detail for illustration only and not by way of limitation. One version of this system has been described (Chien et al. 1991 PNAS USA 88:9578-9582) and is commercially available from Clontech (Palo Alto, Calif.).

Briefly, utilizing such a system, plasmids are constructed that encode two hybrid proteins: one plasmid consists of nucleotides encoding the DNA-binding domain of a transcription activator protein fused to a BDNF receptor (or BDNF) nucleotide sequence encoding BDNF receptor (or BDNF), or a BDNF receptor (or BDNF) polypeptide, peptide or fusion protein, and the other plasmid consists of nucleotides encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, BDNF receptor (or BDNF) may be used as the bait gene product. Total genomic or cDNA sequences are fused to the DNA encoding an activation domain. This library and a plasmid encoding a hybrid of a bait BDNF receptor (or BDNF) gene product fused to the DNA-binding domain are cotransformed into a yeast reporter strain, and the resulting transformants are screened for those that express the reporter gene. For example, and not by way of limitation, a bait BDNF receptor (or BDNF) gene sequence, such as the open reading frame of BDNF receptor or BDNF (or a domain of BDNF receptor) can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait BDNF receptor (or BDNF) gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait BDNF receptor (or BDNF) gene-GAL4 fusion plasmid into a yeast strain, which contains a lacZ gene driven by a promoter that contains GAL4 activation sequence. A cDNA encoded protein, fused to GAL4 transcriptional activation domain, that interacts with bait BDNF receptor (or BDNF) gene product will reconstitute an active GAL4 protein and thereby drive expression of the HIS3 gene. Colonies, which express HIS3, can be detected by their growth on petri dishes containing semi-solid agar based media lacking histidine. The cDNA can then be purified from these strains, and used to produce and isolate the bait BDNF receptor (or BDNF) gene-interacting protein using techniques routinely practiced in the art.

### Assays for Compounds that Interfere with BDNF Receptor/Intracellular or BDNF Receptor/Transmembrane Macromolecule Interaction

The macromolecules that interact with BDNF and BDNF receptor are referred to, for purposes of this discussion, as “binding partners”. These binding partners are likely to be involved in the BDNF receptor signal transduction pathway, and therefore, in the role of BDNF receptor in hippocampal function and verbal memory and risk for neuropsychiatric disorders such as schizophrenia. Therefore, it is desirable to identify compounds that interfere with or disrupt the interaction of such binding partners with BDNF receptor which may be useful in regulating the activity of the BDNF receptor and control hippocampal function and verbal memory and risk for neuropsychiatric disorders such as schizophrenia associated with BDNF receptor activity.

The basic principle of the assay systems used to identify compounds that interfere with the interaction between BDNF and BDNF receptor and its binding partner or partners involves preparing a reaction mixture containing BDNF receptor (or BDNF) protein, polypeptide, peptide or fusion protein as described above, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the BDNF receptor (or BDNF) moiety and its binding partner. Control reaction mixtures are incubated without the test compound or with a placebo. The formation of any complexes between the BDNF receptor (or BDNF) moiety and the binding partner is then detected. The formation of a complex in the control reaction, but not in the reaction mixture containing the test compound, indicates that the compound interferes with the interaction of the BDNF receptor (or BDNF) and the binding partner.

The assay for compounds that interfere with the interaction of the BDNF receptor (or BDNF) and binding partners can be conducted in a heterogeneous or homogeneous format. Heterogeneous assays involve anchoring either the BDNF receptor (or BDNF) moiety product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to or simultaneously with the BDNF receptor (or BDNF) moiety and interactive binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the BDNF receptor (or BDNF) moiety or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the BDNF receptor (or BDNF) gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the BDNF receptor (or BDNF) moiety and the interactive binding partner is prepared in which either the BDNF receptor (or BDNF) or its binding partners is labeled, but the signal generated by the label is quenched due to formation of the complex (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances, which disrupt BDNF receptor (or BDNF)/intracellular binding partner interaction can be identified.

In a particular embodiment, a BDNF receptor (or BDNF) fusion can be prepared for immobilization. For example, the BDNF receptor (or BDNF) or a peptide fragment, e.g., corresponding to a CD, can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art and described above. This antibody can be labeled with the radioactive isotope ¹²⁵I, for example, by methods routinely practiced in the art. In a heterogeneous assay, e.g., the GST-BDNF receptor (or BDNF) fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the BDNF receptor (or BDNF) gene product and the interactive binding partner can be detected by measuring the amount of radioactivity that remains associated with the glutathione-agarose beads. A successful inhibition of the interaction by the test compound will result in a decrease in measured radioactivity.

Alternatively, the GST-BDNF receptor (or BDNF) fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the BDNF receptor (or BDNF)/binding partner interaction can be detected by adding the labeled antibody and measuring the radioactivity associated with the beads.

In another embodiment of the invention, these same techniques can be employed using peptide fragments that correspond to the binding domains of the BDNF receptor (or BDNF) and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

For example, and not by way of limitation, a BDNF receptor (or BDNF) gene product can be anchored to a solid material as described, above, by making a GST-BDNF receptor (or BDNF) fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-BDNF receptor (or BDNF) fusion protein and allowed to bind. After washing away unbound peptides, labeled bound material, representing the intracellular binding partner binding domain, can be eluted, purified, and analyzed for amino acid sequence by well-known methods. Peptides so identified can be produced synthetically or fused to appropriate facilitative proteins using recombinant DNA technology.

### Assays for Identification of Compounds that Modulate Hippocampal Function and Verbal Memory and Risk for Neuropsychiatric Disorders Such as Schizophrenia

Compounds, including but not limited to compounds identified via assay techniques such as those described above, can be tested for the ability to modulate hippocampal function and verbal memory and risk for neuropsychiatric disorders such as schizophrenia. The assays described above can identify compounds that affect BDNF receptor (or BDNF) activity, e.g., compounds that bind to the BDNF receptor (or BDNF), inhibit binding of the natural ligand, BDNF, and either activate signal transduction (agonists) or block activation (antagonists), and compounds that bind to the natural ligand of the BDNF receptor and neutralize ligand activity; or compounds that affect BDNF receptor gene activity (by affecting BDNF receptor gene expression, including molecules, e.g., proteins or small organic molecules, that affect or interfere with events so that expression of the full length BDNF receptor can be modulated). However, it should be noted that the assays described can also identify compounds that modulate BDNF receptor signal transduction (e.g., compounds which affect downstream signaling events, such as inhibitors or enhancers of protein kinases or phosphatases activities which participate in transducing the signal activated by BDNF binding to the BDNF receptor). The identification and use of such compounds which affect another step in the BDNF receptor signal transduction pathway in which the BDNF receptor gene and/or BDNF receptor gene product is involved and, by affecting this same pathway may modulate the effect of BDNF receptor on hippocampal function and verbal memory and risk for neuropsychiatric disorders such as schizophrenia are within the scope of the invention. Such compounds can be used as part of a therapeutic method for impaired hippocampal function and verbal memory and risk for neuropsychiatric disorders such as schizophrenia.

Cell-based systems, membrane vesicle-based systems and membrane fraction-based systems can be used to identify compounds that may act to modulate hippocampal function and verbal memory and risk for neuropsychiatric disorders such as schizophrenia. Such cell systems can include, for example, recombinant or non-recombinant cells, such as cell lines, which express the BDNF receptor (or BDNF) gene. In addition, expression host cells (e.g., COS cells, CHO cells, fibroblasts) genetically engineered to express a functional BDNF receptor (or BDNF) and to respond to activation by the natural ligand, BDNF, e.g., as measured by a chemical or phenotypic change, induction of another host cell gene, change in ion flux (e.g., Ca⁺⁺), phosphorylation of host cell proteins, etc., can be used as an end point in the assay.

In utilizing such cell systems, cells may be exposed to a compound suspected of exhibiting an ability to modulate hippocampal function and verbal memory and risk for neuropsychiatric disorders such as schizophrenia, at a sufficient concentration and for a time sufficient to elicit such a modulation in the exposed cells. After exposure, the cells can be assayed to measure alterations in the expression of the BDNF receptor (or BDNF) gene, e.g., by assaying cell lysates for BDNF receptor (or BDNF) mRNA transcripts (e.g., by Northern analysis) or for BDNF receptor (or BDNF) protein expressed in the cell; compounds which regulate or modulate expression of the BDNF receptor (or BDNF) gene are good candidates as therapeutics. Alternatively, the cells are examined to determine whether one or more cellular phenotypes has been altered to resemble a phenotype more likely to produce a lower incidence or severity of impaired hippocampal function or verbal memory or risk for neuropsychiatric disorders such as schizophrenia. Still further, the expression and/or activity of components of the signal transduction pathway of which BDNF receptor is a part, or the activity of the BDNF receptor signal transduction pathway itself can be assayed.

For example, after exposure, the cell lysates can be assayed for the presence of phosphorylation of host cell proteins, as compared to lysates derived from unexposed control cells. The ability of a test compound to inhibit phosphorylation of host cell proteins in these assay systems indicates that the test compound alters signal transduction initiated by BDNF receptor activation. The cell lysates can be readily assayed using a Western blot format; i.e., the host cell proteins are resolved by gel electrophoresis, transferred and probed using a detection antibody (e.g., an antibody labeled with a signal generating compound, such as radiolabel, fluor, enzyme, etc.). (See, e.g., Glenney et al. 1988 J Immunol Methods 109:277-285; Frackelton et al. 1983 Mol Cell Biol 3:1343-1352). Alternatively, an ELISA format could be used in which a particular host cell protein involved in the BDNF receptor signal transduction pathway is immobilized using an anchoring antibody specific for the target host cell protein, and the presence or absence of a phosphorylated residue on the immobilized host cell protein is detected using a labeled antibody. (See, King et al. 1993 Life Sci 53:1465-1472). In yet another approach, ion flux, such as calcium ion flux, can be measured as an end point for BDNF receptor stimulated signal transduction.

In addition, animal-based memory disorder models, which may include, for example, transgenic mice as described above, may be used to identify compounds capable of modulating hippocampal function and verbal memory and risk for neuropsychiatric disorders such as schizophrenia. Such animal models may be used as test substrates for the identification of drugs, pharmaceuticals, therapies and interventions which may be effective in treating impaired hippocampal function or verbal memory or neuropsychiatric disorders such as schizophrenia. For example, animal models may be exposed to a compound, suspected of exhibiting an ability to modulate hippocampal function or verbal memory or neuropsychiatric disorders such as schizophrenia, at a sufficient concentration and for a time sufficient to elicit such a modulation of hippocampal function or verbal memory or neuropsychiatric disorders such as schizophrenia in the exposed animals. The response of the animals to the exposure may be monitored by assessing their performance of memory tasks. With regard to intervention, any treatments which reverse any aspect of impaired hippocampal function or verbal memory or neuropsychiatric disorders such as schizophrenia should be considered as candidates for human therapeutic intervention. Dosages of test agents may be determined by deriving dose-response curves.

### Pharmaceutical Preparations and Methods of Administration

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably Within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically acceptable excipients. Thus, the compounds and their physiologically acceptable salts and solvates may be formulated for administration by, for example, injection, inhalation or insufflation (either through the mouth or the nose), or oral, buccal, parenteral or rectal administration, or through molecular techniques using gene therapy.

For such therapy, the compounds of the invention can be formulated for a variety of loads of administration, including systemic and topical or localized administration. Techniques and formulations generally may be found in Reminington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possibility of local noninvasive delivery of drugs over an extended period of time. This technology utilizes microspheres of precapillary size which can be injected via a catheter into any selected part of the e.g. brain or other organs. The administered therapeutic is slowly released from these microspheres and taken up by surrounding tissue cells.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the compounds of the invention are formulated into ointments, salves, gels, or creams as generally known in the art.

In clinical settings, a gene delivery system for a nucleotide sequence encoding an antisense, ribozyme or dominant negative mutant can be introduced into a patient by any of a number of methods, each of which is familiar in the art. For instance, a pharmaceutical preparation of the gene delivery system can be introduced systemically, e.g., by intravenous injection, and specific transduction of the gene in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the gene is more limited with introduction into the individual being quite localized. For example, the gene delivery vehicle can be introduced by catheter (see U.S. Pat. No. 5,328,470) or by stereotactic injection (e.g., Chen et al. 1994 PNAS USA 91:3054-3057).

The pharmaceutical preparation of the gene therapy construct or compound of the invention can consist essentially of the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. A method for predicting the likelihood that an individual will have impaired or enhanced hippocampal function, comprising the steps of obtaining a DNA sample from an individual to be assessed and determining the presence or absence of a single nucleotide polymorphism from G to A resulting in the substitution of a methionine residue for a valine residue at amino acid position 66, relative to the start of the precursor protein sequence for brain-derived neurotrophic factor (BDNF), wherein a single nucleotide polymorphism from G to A resulting in the substitution of a methionine residue for a valine residue at amino acid position 66 (relative to the start of the precursor protein sequence) is correlated with impaired hippocampal function, and a single nucleotide polymorphism from A to G resulting in the substitution of a valine residue for a methionine residue at amino acid position 66 (relative to the start of the precursor protein sequence) is correlated with enhanced hippocampal function.

2. A method for predicting the likelihood that an individual will have impaired or enhanced verbal memory, comprising the steps of obtaining a DNA sample from an individual to be assessed and determining the presence or absence of a single nucleotide polymorphism from G to A resulting in the substitution of a methionine residue for a valine residue at amino acid position 66, relative to the start of the precursor protein sequence for brain-derived neurotrophic factor (BDNF), wherein a single nucleotide polymorphism from G to A resulting in the substitution of a methionine residue for a valine residue at amino acid position 66 (relative to the start of the precursor protein sequence) is correlated with impaired verbal memory, and a single nucleotide polymorphism from A to G resulting in the substitution of a valine residue for a methionine residue at amino acid position 66 (relative to the start of the precursor protein sequence) is correlated with enhanced verbal memory.

3. A method for predicting the likelihood that an individual has risk for or protection from schizophrenia, schizoaffective disorder, and other psychotic and mental disorders involving impaired memory, comprising the steps of obtaining a DNA sample from an individual to be assessed and determining the presence or absence of a single nucleotide polymorphism from G to A resulting in the substitution of a methionine residue for a valine residue at amino acid position 66, relative to the start of the precursor protein sequence for brain-derived neurotrophic factor (BDNF), wherein a single nucleotide polymorphism from G to A resulting in the substitution of a methionine residue for a valine residue at amino acid position 66 (relative to the start of the precursor protein sequence) is correlated with risk for schizophrenia, schizoaffective disorder, and other psychotic and mental disorders involving impaired memory, and a single nucleotide polymorphism from A to G resulting in the substitution of a valine residue for a methionine residue at amino acid position 66 (relative to the start of the precursor protein sequence) is correlated with protection from schizophrenia, schizoaffective disorder, and other psychotic and mental disorders involving impaired memory.

4. A method according to claim 1, wherein the individual is an individual at risk for development of impaired hippocampal function.

5. The method according to claim 2, wherein the individual is an individual at risk for development of impaired verbal memory.

6. A method according to claim 3, wherein the individual is an individual at risk for development of schizophrenia, schizoaffective disorder, and other psychotic and mental disorders involving impaired memory.

7. A method according to claim 1, wherein the individual exhibits clinical symptomatology associated with impaired hippocampal function.

8. A method according to claim 2, wherein the individual exhibits clinical symptomatology associated with impaired verbal memory.

9. A method according to claim 3, wherein the individual exhibits clinical symptomatology associated with schizophrenia, schizoaffective disorder, and other psychotic and mental disorders involving impaired memory.

10. A method according to claim 1, wherein the individual has been clinically diagnosed as having impaired hippocampal function.

11. A method according to claim 2, wherein the individual has been clinically diagnosed as having impaired verbal memory.

12. A method according to claim 3, wherein the individual has been clinically diagnosed as having schizophrenia, schizoaffective disorder, and other psychotic and mental disorders involving impaired memory.

13. A method for predicting the likelihood than an individual will have impaired or enhanced hippocampal function, comprising the steps of obtaining a biological sample from an individual to be assessed containing the precursor BDNF protein or relevant portion thereof and determining the amino acid present at amino acid position +66 relative to the first amino acid of the precursor protein, wherein the presence of methionine at this position is indicative of impaired hippocampal function, and the presence of valine at this position is indicative of enhanced hippocampal function.

14. A method for predicting the likelihood than an individual will have impaired or enhanced verbal memory, comprising the steps of obtaining a biological sample from an individual to be assessed containing the precursor BDNF protein or relevant portion thereof and determining the amino acid present at amino acid position +66 relative to the first amino acid of the precursor protein, wherein the presence of methionine at this position is indicative of impaired verbal memory, and the presence of valine at this position is indicative of enhanced verbal memory.

15. A method for predicting the likelihood than an individual will have risk for or protection from schizophrenia, schizoaffective disorder, and other psychotic and mental disorders involving impaired memory, comprising the steps of obtaining a biological sample from an individual to be assessed containing the precursor BDNF protein or relevant portion thereof and determining the amino acid present at amino acid position +66 relative to the first amino acid of the precursor protein, wherein the presence of methionine at this position is indicative of risk for schizophrenia, schizoaffective disorder, and other psychotic and mental disorders involving impaired memory, and the presence of valine at this position is indicative of protection from schizophrenia, schizoaffective disorder, and other psychotic and mental disorders involving impaired memory.

16. A method according to claim 13, wherein the individual is an individual at risk for development of impaired hippocampal function.

17. The method according to claim 14, wherein the individual is an individual at risk for development of impaired verbal memory.

18. A method according to claim 15, wherein the individual is an individual at risk for development of schizophrenia, schizoaffective disorder, and other psychotic and mental disorders involving impaired memory.

19. A method according to claim 13, wherein the individual exhibits clinical symptomatology associated with impaired hippocampal function.

20. A method according to claim 14, wherein the individual exhibits clinical symptomatology associated with impaired verbal memory.

21. A method according to claim 15, wherein the individual exhibits clinical symptomatology associated with schizophrenia, schizoaffective disorder, and other psychotic and mental disorders involving impaired memory.

22. A method according to claim 13, wherein the individual has been clinically diagnosed as having impaired hippocampal function.

23. A method according to claim 14, wherein the individual has been clinically diagnosed as having impaired verbal memory.

24. A method according to claim 15, wherein the individual has been clinically diagnosed as having schizophrenia, schizoaffective disorder, and other psychotic and mental disorders involving impaired memory.

25. A method for screening compounds useful for modulation of hippocampal function, comprising the steps of contacting a compound with a cultured host cell or membrane thereof that expresses a BDNF receptor or domain thereof and detecting binding of said compound to the BDNF receptor for domain thereof, whereby said binding identifies said compound as a candidate useful for modulation of hippocampal function.

26. The method of claim 25, which further comprises conducting the identification of the compound in the presence of labeled or unlabeled BDNF or homolog thereof.

27. A method for screening compounds useful for modulation of verbal memory, comprising the steps of contacting a compound with a cultured host cell or membrane thereof that expresses a BDNF receptor or domain thereof and detecting binding of said compound to the BDNF receptor for domain thereof, whereby said binding identifies said compound as a candidate useful for modulation of verbal memory.

28. The method of claim 27, which further comprises conducting the identification of the compound in the presence of labeled or unlabeled BDNF or homolog thereof.

29. A method for screening compounds useful for modulation of risk for schizophrenia, schizoaffective disorder, and other psychotic and mental disorders involving impaired memory, comprising the steps of contacting a compound with a cultured host cell or membrane thereof that expresses a BDNF receptor or domain thereof and detecting binding of said compound to the BDNF receptor for domain thereof, whereby said binding identifies said compound as a candidate useful for modulation of risk for schizophrenia, schizoaffective disorder, and other psychotic and mental disorders involving impaired memory.

30. The method of claim 29, which further comprises conducting the identification of the compound in the presence of labeled or unlabeled BDNF or homolog thereof.

31. A method for screening compounds useful for modulation of hippocampal function, comprising the steps of contacting a compound with a cultured host cell or membrane thereof that expresses a BDNF receptor or domain thereof, in the presence of labeled or unlabeled BDNF or homolog thereof, and determining whether said compound changes binding of said BDNF or homolog thereof to said BDNF receptor or domain thereof by measuring an amount of said BDNF or homolog thereof bound to said BDNF receptor or domain thereof, and identifying said compound as a candidate useful for modulation of hippocampal function, whereby said compound causes a change in binding of said BDNF or homolog thereof.

32. A method for screening compounds useful for modulation of verbal memory, comprising the steps of contacting a compound with a cultured host cell or membrane thereof that expresses a BDNF receptor or domain thereof, in the presence of labeled or unlabeled BDNF or homolog thereof, and determining whether said compound changes binding of said BDNF or homolog thereof to said BDNF receptor or domain thereof by measuring an amount of said BDNF or homolog thereof bound to said BDNF receptor or domain thereof, and identifying said compound as a candidate useful for modulation of verbal memory, whereby said compound causes a change in binding of said BDNF or homolog thereof.

33. A method for screening compounds useful for modulation of risk for schizophrenia, schizoaffective disorder, and other psychotic and mental disorders involving impaired memory, comprising the steps of contacting a compound with a cultured host cell or membrane thereof that expresses a BDNF receptor or domain thereof, in the presence of labeled or unlabeled BDNF or homolog thereof, and determining whether said compound changes binding of said BDNF or homolog thereof to said BDNF receptor or domain thereof by measuring an amount of said BDNF or homolog thereof bound to said BDNF receptor or domain thereof, and identifying said compound as a candidate useful for modulation of risk for schizophrenia, schizoaffective disorder, and other psychotic and mental disorders involving impaired memory, whereby said compound causes a change in binding of said BDNF or homolog thereof.

34. A method for screening compounds useful for modulation of hippocampal function, comprising the steps of contacting a compound with a transgenic animal that expresses an exogenous BDNF gene and/or has one or both alleles of an endogenous BDNF gene inactivated, and detecting a change in said transgenic animal, whereby said change identifies said compound as a candidate useful for modulation of hippocampal function.

35. The method of claim 34, wherein the transgenic animal is a transgenic mouse.

36. A method for screening compounds useful for modulation of verbal memory, comprising the steps of contacting a compound with a transgenic animal that expresses an exogenous BDNF gene and/or has one or both alleles of an endogenous BDNF gene inactivated, and detecting a change in said transgenic animal, whereby said change identifies said compound as a candidate useful for modulation of verbal memory.

37. The method of claim 36, wherein the transgenic animal is a transgenic mouse.

38. A method for screening compounds useful for modulation of risk for schizophrenia, schizoaffective disorder, and other psychotic and mental disorders involving impaired memory, comprising the steps of contacting a compound with a transgenic animal that expresses an exogenous BDNF gene and/or has one or both alleles of an endogenous BDNF gene inactivated, and detecting a change in said transgenic animal, whereby said change identifies said compound as a candidate useful for modulation of risk for schizophrenia, schizoaffective disorder, and other psychotic and mental disorders involving impaired memory.

39. The method of claim 38, wherein the transgenic animal is a transgenic mouse.

40. A method of making a pharmaceutical composition useful for modulation of hippocampal function, comprising combining a pharmaceutically acceptable excipient and a compound identified by any of the preceding screening methods of claims 25, 26, 31, 34, or 35.

41. A method of making a pharmaceutical composition useful for modulation of verbal memory, comprising combining a pharmaceutically acceptable excipient and a compound identified by any of the preceding screening methods of claims 27, 28, 32, 36, or 37.

42. A method of making a pharmaceutical composition useful for modulation of risk for schizophrenia, schizoaffective disorder, and other psychotic and mental disorders involving impaired memory, comprising combining a pharmaceutically acceptable excipient and a compound identified by any of the preceding screening methods of claims 29, 30, 33, 38, or 39.

43. A method of modulating hippocampal function in an individual, comprising administering a compound to the individual in an amount sufficient to mimic or inhibit binding of a BDNF receptor by endogenous BDNF, whereby hippocampal function is modulated.

44. A method of modulating verbal memory in an individual, comprising administering a compound to the individual in an amount sufficient to mimic or inhibit binding of a BDNF receptor by endogenous BDNF, whereby verbal memory is modulated.

45. A method of modulating risk for schizophrenia, schizoaffective disorder, and other psychotic and mental disorders involving impaired memory in an individual, comprising administering a compound to the individual in an amount sufficient to mimic or inhibit binding of a BDNF receptor by endogenous BDNF, whereby risk for schizophrenia, schizoaffective disorder, and other psychotic and mental disorders involving impaired memory is modulated.

46. A method of modulating hippocampal function in an individual, comprising administering a compound identified/by any of the preceding screening methods of claims 25, 26, 31, 34, or 35 to the individual in an amount sufficient to modulate hippocampal function.

47. A method of modulating verbal memory in an individual, comprising administering a compound identified by any of the preceding screening methods of claims 27, 28, 32, 36, or 37 to the individual in an amount sufficient to modulate verbal memory.

48. A method of modulating risk for schizophrenia, schizoaffective disorder, and other psychotic and mental disorders involving impaired memory in an individual, comprising administering a compound identified by any of the preceding screening methods of claims 29, 30, 33, 38, or 39 to the individual in an amount sufficient to modulate risk for schizophrenia, schizoaffective disorder, and other psychotic and mental disorders involving impaired memory.

49. A pharmaceutical composition, comprising a compound identified by any of the preceding screening methods of claims 25, 26, 31, 34, or 35 in combination with a pharmaceutically acceptable excipient.

50. A pharmaceutical composition, comprising a compound identified by any of the preceding screening methods of claims 27, 28, 32, 36, or 37 in combination with a pharmaceutically acceptable excipient.

51. A pharmaceutical composition, comprising a compound identified by any of the preceding screening methods of claims 29, 30, 33, 38, or 39 in combination with a pharmaceutically acceptable excipient.
